(19) United States
(12) Patent Application Publication
(10) Pub. No.: US 2023/0263901 A1
(43) Pub. Date: Aug. 24, 2023

(54) MUCUS ADHESION DRUG DELIVERY

(71) Applicant: Yimin Sun, Zhaoqing, Guangdong (CN)

(72) Inventor: Yimin Sun, Zhaoqing, Guangdong (CN)

(21) Appl. No.: 18/040,315

(22) PCT Filed: Jan. 29, 2021
(86) PCT No.: PCT/CN2021/074312

§ 371 Date: Feb. 2, 2023

(51) Int. Cl. A61K 47/61

(52) U.S. Cl. CPC A61K 47/61

(57) ABSTRACT

The present invention relates to a mucus adhesion drug delivery technology. The technology involves the preparation of materials consisting of mucus adhesive excipient polymer chemically bonded with drug molecules and delivery of the materials with fast disintegration tablet with enteric coating to intestine. The bonding between the excipient polymer and the drug molecules is either ionic bonds, covalent bonds, or metal coordination bonds. The test of the mucus adhesion drug delivery technology was carried out using a dog model. The test data indicated successful retaining of drug molecules on the intestine mucus resulting in not only dramatic extension of drug release time, but also great improvement in the bioavailability of the API.

### BACKGROUND OF INVENTION

### 1. Field of Invention

The present invention relates to mucus adhesion drug delivery.

### 2. Description of Prior Art

Utilization of mucus adhesive excipient to prolong retention of drug molecules in human gastric tract was attempted long time ago. The experimental data showed that success was very limited. For drug molecules with short half-life (<2 hr) extended drug release more than 6 hours without losing bioavailability seems very rare, almost impossible to achieve.

A human GI tract consists of three major parts: stomach, intestine, and colon. For most of drug products, the absorption of drug molecules occurs at intestine. When food is ingested, it usually stays in stomach for up to about 45 minutes in average. Then it will take approximate 4 hours to pass through intestine. Therefore, for any drug molecules with short half-life (<2 hours), drug concentration in blood will generally drop below the therapeutic concentration after 6 hours. Any extended-release formulation longer than 6 hours involving drug molecules with short half-life, will miss the absorption window intestine resulting in reduced bioavailability. Scientists have tried very hard to extend the drug absorption window by retention of drug molecules with a mucus adhesive delivery vehicle. There is no doubt about the mucus adhesion capability of some of the polymer excipients, however, until today there is little experimental evidence to prove the retention of drug molecules via mucus adhesive excipient.

For example, a dosage form can keep releasing drug molecules up to 12 hours or 24 hours when testing in a dissolution tester in the lab. Therefore, some people claimed that their dosage form were extended release based on their laboratory dissolution test results. But this does not mean that their dosage forms can actually achieve extended drug absorption in a human body or an animal model. As stated above, there is drug absorption window (intestine) in GI track. True extended drug delivery can only be tested and confirmed in an animal model. To the best knowledge of the inventor, currently no alternative in vitro laboratory testing machine can prove extended drug delivery via mucus adhesion.

Matrix based extended drug delivery dosage forms in the art are most likely to fail in achieving mucus adhesion because the specific surface area of the matrix is too small to retain on mucus. In another word, only particles small enough can retain on mucus effectively. That means tablets must be disintegrated into small pieces to achieve effective mucus adhesion. That is exactly the immediate release dosage form. However, once the tablet breaks up into small pieces, the excipient will have no control over the movement of API. API cannot retain on mucus. This seems to be a contradiction and unsolvable challenge in the art.

The mucus adhesion is a surface phenomenon governed by surface chemistry principles. To adhere to a surface, particles must have a specific surface area large enough. In another word, the particles size must be small enough for adhesion to occur. The smaller the particle size the stronger the adhesion. This is an insurmountable challenge for conventional formulations. Most extended-release formulations are matrix based. The excipient gel matrix holds the API crystals. The crystals dissolve slowly and defuse out of matrix to achieve controlled release of drug molecules. Usually, the matrix sizes are quite large ranging from a few mm to a few cm, rendering very weak mucus adhesion of the matrix with the mucus surfaces.

For fast disintegration formulations, there is no matrix. The particle size of excipients is small enough to attach to the surface of mucus. But there is no bonding between API crystals and mucus adhesive excipients, rendering the retention of drug molecules to the mucus surface impossible. Therefore, conventional formulation technology cannot achieve retention of drug molecules to the mucus surface via mucus adhesive polymer excipients effectively.

### SUMMARY OF THE INVENTION

The first aspect of the present application is a composition comprising an effective amount of a biologically active agent bonded with a mucoadhesive polymer ionically or covalently or via metal coordination bonding. After the composition is administered to a subject, the biologically active agent exerts its pharmaceutical effect while retaining on mucus of the animal.

The effective amount of biologically active agent bonded with a mucoadhesive polymer ionically or covalently or via metal coordination bonding may be compressed into tablets with other excipients, and the tablets may be coated with an enteric coating material so that the pharmaceutically active compound is delivered to intestine, gradually released in an intestine of the subject and absorbed through an intestine wall into blood stream of the subject in presence of NaCl or other ionic compound or via hydrolysis in the body fluid of the subject.

The biologically active agent may comprise at least one functional group selected from the group consisting of amine, carboxyl, hydroxyl, sulfonic functional group —SO₃H, cationic metal, and combinations thereof, and the mucoadhesive polymer may comprise at least one corresponding functional group selected from the consisting of carboxyl, hydroxyl, amine, sulfonic functional group —SO₃H, and combinations thereof so that the pharmaceutically active compound is bonded with the mucoadhesive polymer ionically or covalently or via metal coordination.

Preferably, the biologically active agent is ionically bonded with the mucoadhesive polymer, and the pharmaceutically active compound is released in the body of the animal via metastasis. The biologically active agent preferably comprises at least one functional group selected from the group consisting of amine, carboxyl, sulfonic functional group —SO₃H, and combinations, and the mucoadhesive polymer preferably comprises at least one corresponding functional group selected from the consisting of carboxyl, amine, sulfonic functional group —SO₃H, and combinations.

The biologically active agent may also be covalently bonded with the mucoadhesive polymer, and the pharmaceutically active compound is released in the body of the animal via hydrolysis.

The mucoadhesive polymer may be selected from the group consisting of poly(methylvinylether co-methacrylic acid), poly(acrylic acid-co ethylhexylacrylate), copolymer of acrylic acid and poly ethylene glycolpolymer, copolymer of acrylic acid or methacrylic acid, carbopol, polycarbophil, carbomer, pectin, alginic acid, hyaluronic acid, chitosan, tragacanth gum, karaya gum, xanthan gum, carboxymethylcellulose, polyvinylamine, polyallylamine, polyethyleneimine, and combinations thereof.

The biologically active agent may be selected from the group consisting of ibuprofen, lanthnum chloride, levodopa ethyl ester, aspirin, glutathione, valsartan, quetiapine, duloxetine, oxymorphone, and combinations thereof.

The biologically active agent may also be a biologically active agent with a short half-life (<2 hrs) or low bioavailability (< 80%) due to missing out absorption window of GI tract, and the composition has an extended time of delivery of the biologically active agent for more than 12 hours.

The composition of the present application may comprise less than 50%, preferably less than 30% by molar amount of the biologically active agent in free form relative to the biologically active agent bonded with the mucoadhesive polymer by molar amount.

The biologically active agent bonded with the mucoadhesive polymer has a diameter of less than 3 mm, and preferably a broad particle size distribution.

Compared to a conventional formulation in the art, which comprises a free form of a biologically active agent not chemically bonded with a mucoadhesive polymer as a primary active agent, the composition of the present application preferably has a delayed release profile and/or an improved bioavailability.

The second aspect of the present application is a method of extending release time of a biologically active agent and increase bioavailability of the biologically active agent in a subject comprising:
preparing the composition of the present application; and
administering the composition to a subject in an effective amount.

The administering may comprise delivering the composition through mucosa of mouth, nose, stomach, intestine, and/or or eye of the subject.

The third aspect of the present application is a method of making the composition of the present application comprising: reacting an effective amount of the biologically active agent with mucoadhesive polymer in water or a non-aqueous solvent via acid-base reaction, metathesis reaction, or esterification reaction to make the pharmaceutically active compound bonded with the mucoadhesive polymer. For example, when the biologically active agent is LaCl₃, the method may comprise reacting an effective amount of LaCl₃ with a mucoadhesive polymer in water and/or a non-aqueous solvent via metathesis reaction to make LaCl₂ bonded with the mucoadhesive polymer via metal coordination. The method may also comprise reacting an effective amount of the biologically active agent with a mucoadhesive polymer in a non-aqueous solvent via esterification reaction to make the pharmaceutically active compound bonded with the mucoadhesive polymer via ester covalent bonding.

### BRIEF DESCRIPTION OF THE DRAWING

FIG. 1 shows formation of ionic bonding between ibuprofen and chitosan, mucus adhesion, and release of free drug molecules via metathesis in the presence of body fluid.

FIG. 2 shows formation of mucus adhesive levodopa ethyl ester/hyaluronic acid material and release of free drug molecules via metathesis in the presence of body fluid.

FIG. 3 shows formation of mucus adhesive lanthnum chloride/hyaluronic acid material and capture of phosphate ions via metathesis in the presence of body fluid.

FIG. 4 shows ibuprofen blood concentration vs. time profile based on examples described below.

FIG. 5 shows levodopa blood concentration vs. time profile based on examples described below.

### DETAILED DESCRIPTION OF PREFERRED EMBODIMENTS OF THE INVENTION

The following embodiments are provided to illustrate, but not to limit the instant invention.

As used herein and in the appended claims, “a,” “an,” and “the” include plural referents unless the context clearly dictates otherwise.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

The terms “subject” and “patient” are used interchangeably. In some embodiments, the subject is a mammal. In some embodiments, the subject is a human. In some embodiments, the subject is an animal, such as a mouse, rat, rabbit, dog, donkey, or a laboratory test animal, etc.

Biologically active agent suitable for incorporation in a composition according to the present invention include medicaments, drugs, nutrients, or other suitable biologically, physiologically, diagnostically, or pharmaceutically active substances capable of providing a local or systemic biological, physiological, diagnostical, or therapeutic effect. Non-limiting examples of therapeutic effects are antimetabolic, antifungal, anti-inflammatory, antihypertensive, antipsychotic, analgesic, antidiabetic, hypnotic, sedating, anesthetic, antitumoral, antiinfectious, antibiotic, antiviral, hormonal, nutrient, agonist, and antagonist properties. For example, the biologically active agents may include ibuprofen, aspirin, glutathione, valsartan, quetiapine, duloxetine, and oxymorphone. For another example, a biologically active agent LaCl₃ salt itself may have no pharmaceutical effects, it can bind with phosphate ions in a patient to prevent phosphate ions in food to be absorbed into blood. Thus, it can maintain the phosphate level in blood relatively low for a patient whose kidney has lost its function to regulate phosphate. Keeping LaCl₂ on the mucus to trap phosphate ions can prevent phosphate ions to be absorbed into blood. The purpose of using mucus adhesion LaC³₃ is to reduce the quantity of LaCl₃ needed to regulate phosphate ion level, thus reducing the adverse effects caused by LaCl₃ to the GI tract.

In general, enteric coating materials used in the present application are designed to prevent premature drug release from an oral solid dosage form in the low pH environment of the stomach, thereby delaying drug release until the dosage form reaches the small intestine. The enteric coating materials may be selected from those commercially available materials. The enteric coating materials may comprise, as an example, a methacrylic acid copolymer or multiple types of methacrylic acid copolymers. The preferred enteric materials are shellac (esters of aleurtic acid), cellulose acetate phthalate (CAP), poly(meth-acrylic acid-co-methyl methacrylate ), poly(methacrylic 65 acid-co-ethyl methacrylate ), cellulose acetate trimellitate (CAT), poly(vinyl acetate phthalate) (PVAP), hydroxypropyl methylcellulose phthalate (HPMCP) and hydroxypropyl methylcellulose acetate succinates. The preferred enteric polymers release at a pH of greater than or equal to pH 5.5. Examples include any of Eudragit® L 30 D-55 (poly(methacrylic acid-co-ethyl acrylate) 1:1; CAS Number 25212-88-8; Evonik Industries), Eudragit® L 100-55 (poly(methacrylic acid-co-ethyl acrylate) 1:1; CAS Number 25212-88-8; Evonik Industries), Eudragit® L 100 (poly(methacrylic acid-co-methyl methacrylate) 1: 1; CAS Number 25086-15-1; Evonik Industries), Eudragit® L 12,5 (poly(methacrylic acid-co-methyl methacrylate) 1:1; CAS Number 25086-15-1; Evonik Industries); Eudragit® S 100 (poly(methacrylic acid-co-methyl methacrylate) 1:2; CAS Number 25086-15-1; Evonik Industries), Eudragit® S 12,5 (poly(methacrylic acid-co-methyl methacrylate) 1:2; CAS Number 25086-15-1; Evonik Industries), and Eudragit® S 30 D (poly( methacrylate-co-methyl methacrylate-co-meth-acrylic acid) 7:3:1; CAS Number 26936-24-3; Evonik Industries) or a combination thereof. The enteric coating materials may constitute 2-20% of the mass of the composition, preferably 3-15%, most preferably 5-12%.

The composition of the present application may be made in any suitable form, such as tablets, cachets, lozenges, capsules, solution, and suspension, preferably an oral solid dosage, such as tablets.

The composition of the present application may attach to any mucus surfaces, inner nose mucus, buccal mucus, eyes, stomach, intestine, etc. to achieve extended drug release. Attaching the composition onto stomach may produce extended drug delivery and shorten the delay of drug release profile. Nose and buccal delivery may be especially useful to deliver those drugs with extremely low bioavailability when administered via oral solid dosage due to first pass elimination. The composition of the present application may be delivered to a subject or patient through mucosa of mouth, nose, stomach, intestine, and/or or eye.

Generally, it is easier to make a 12-hour extended-release dosage form for a drug molecule whose half-life is more than 6 hours because even immediate release dosage form will have an approximate 5-6-hour drug delivery time as explained above and plus >6 hours half-life will easily produce a 12-hour drug release profile. However, it is very challenging to deliver 12-hour drug supply for a drug with a half-life of only 2 hours because 6+2=8 hours. The shorter the half-life of the drug is, the more challenge to achieve extended drug delivery of greater than 5-6 hours. The mucus adhesive drug delivery of the present application may not only deliver drugs with longer than 2 hours half-life. More importantly our mucus adhesive drug delivery can extend the drug delivery to more than 12 hours for the drugs with half-life <2 hrs.

The composition of the present application may also comprise one or more additives, e.g., nonionic auxiliary substances such as diluents, carriers, excipients, or stabilizers. For example, the composition of the present application may comprise lactose and/or microcrystalline cellulose as diluents and crosslinked sodium carboxymethyl cellulose as disintegrant. According to one embodiment of the present application, the composition is in a fast disintegration form and comprises a non-ionic filler excipient and a disintegrant. The molar drug load can be 0-100%, preferably 20-80%, most preferably 40-60%.

The mucoadhesive polymer of the present application can be any suitable polymer. For example, it may be polymer or copolymer of acrylic acid or methacrylic acid, pectin, alginic acid, hyaluronic acid, chitosan, tragacanth gum, karaya gum, xanthan gum, carboxymethylcellulose, polyvinylamine, polyallylamine, polyethyleneimine, carbopol, polycarbophil, poly(methylvinylether co-methacrylic acid), poly(acrylic acid-co ethylhexylacrylate), copolymer of acrylic acid and poly ethylene glycol. The examples are chitosan and hyaluronic acid. The molecule weight of the polymers ranges from 500-5,000,000, which may be chosen based on the need of mucus adhesion. For example, the molecular weight of hyaluronic acid is 1.3 million. The viscosity of chitosan is 138mpa.s at 20° C. with concentration of 10 g/L H₂O.

The biologically active agent chemically bonded with a mucoadhesive polymer is substantially different from a simple physical admixture of a biologically active agent and a mucoadhesive polymer, which are not associated with each other via chemical reaction. The biologically active agent chemically bonded with a mucoadhesive polymer in the composition of the present invention are not free molecules and these bonded molecules may not freely contact the receptors of the GI tract or other sites to generate a biological response. Therefore, the composition of the present invention may be used for taste masking for buccal drug delivery. Moreover, the biological adverse effect manifested in the GI tract of the composition of present invention may be substantially reduced comparing with a physical admixture of a biologically active agent and a mucoadhesive polymer. Moreover, the biologically active agent chemically bonded with a mucoadhesive polymer may offer a complete solution to low absorption problem due to low solubility of the drug because the drug molecules in the composition exists in an individual molecular state.

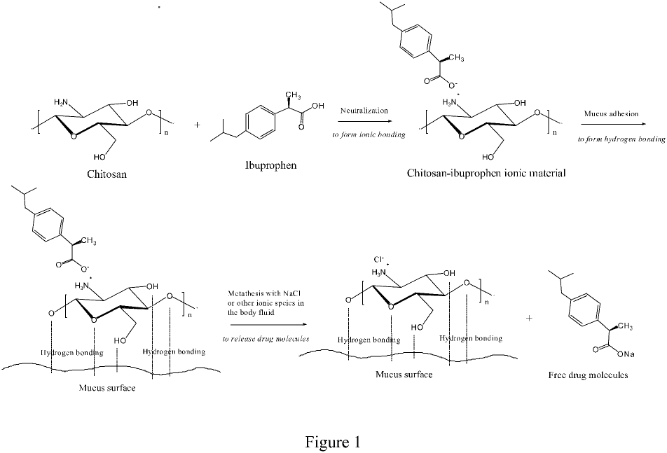

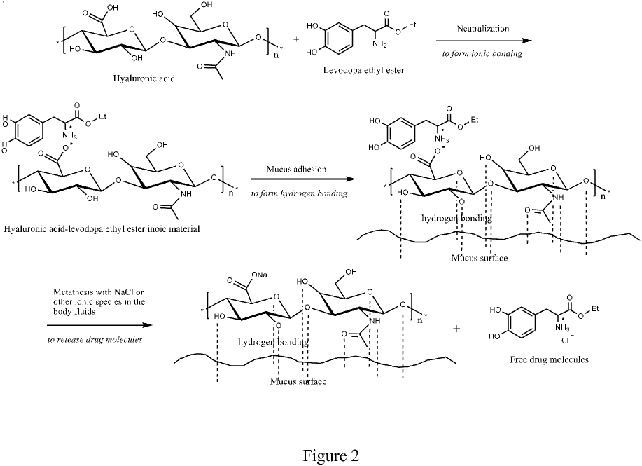

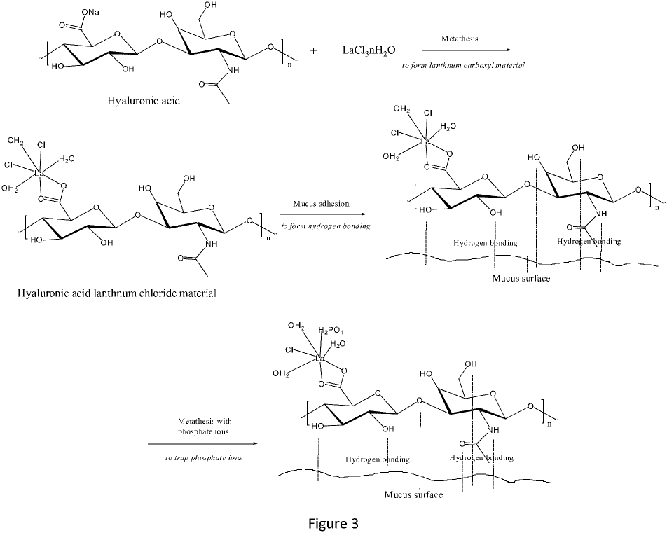

The present inventor has made intensive research to develop an innovative drug delivery technology to overcome the shortcomings of the existing extended drug delivery methods. The technology comprises synthesis of a mucus adhesive excipient polymer chemically bonded with drug molecules and delivery of the mucus adhesive material to intestine mucus wall. To prevent interference of gastric fluid and maximize the mucus adhesion, a fast disintegration solid dosage form with enteric coating was developed. Ibuprofen and levodopa ethyl ester were selected as examples of carboxy-containing compounds and amine-containing compounds, respectively. See FIGS. 1 and 2. Both compounds have relative short half-life. Dog model was used to test the drug delivery technology. It is noticed that the ibuprofen and levodopa ethyl ester drug delivery data clearly demonstrated that the mucus adhesion drug delivery technology greatly extended drug delivery time and greatly enhanced bioavailability at the same time vs. conventional formulation technology, indicating the mucus adhesion of drug molecules via mucus adhesive excipient polymer. The unique feature of this mucus adhesion drug delivery technology is the simultaneous increase in drug release time and in bioavailability of the drug.

According to an embodiment of the present application, a mucus adhesive material comprising mucus adhesive excipient polymers bearing amine function groups, —NH₂, and drug molecules bearing a corresponding carboxyl function group, —COOH is made. See, for example, see FIG. 1 for illustration.

According to another embodiment of the present application, a mucus adhesive material comprising mucus adhesive excipient polymers bearing carboxyl function groups, —COOH, and drug molecules bearing amine groups, —NH₂, is made. See, for example, FIG. 2 for illustration.

According to yet another embodiment of the present application, there is provided a formulation scheme using the mucus adhesive material comprising mucus adhesive excipient polymer bonded with drug molecules. A fast disintegration tablet formulation is preferable. The enteric coating of the tablet is preferable to prevent the interference of the gastric fluid with the integrity of the mucus adhesive material bonded with drug molecules.

According to a further embodiment of the present application, there is provided a dog model drug delivery tests to confirm the validity of mucus adhesion drug delivery. The test results have showed not only substantial increase in drug delivery time but also great enhancement in bioavailability vs. conventional drug delivery. The simultaneous increase in both drug delivery time and bioavailability is a strong evidence of achieving mucus adhesion of drug molecules.

### EXAMPLES

### Example 1: Preparation of Mucus Adhesive Ibuprofen/Chitosan Material (NW1901-82)

Added chitosan (2.02 g) to water (140 mL). To the slurry added 1.0 N HCl (10 mL). Stirred for 30 minutes and a clear solution was obtained. Diluted 1.0 N NaOH (10 mL) to 40 mL and dropwise added to the above chitosan solution. After stirring for 1 hour, separated chitosan polymer by centrifuging. Washed the polymer with water and hydrated chitosan (64.0 g) was obtained. Added water (50 mL) and ibuprofen (0.99 g) and stirred for 3 hours. Added 1.0 N HCl (6.0 mL), stirred for 2 hours, added lactose (1.25 g) and stirred for 1 hour. Evaporated water in 65° C. oven. Solid material (4.25 g) was obtained and milled into powder with pestle and mortar.

### Example 2: Formulation of Mucus Adhesive Ibuprofen/Chitosan (NW1901-84)

Weighed ibuprofen/chitosan material (3.831 g) made based on Example 1 and lactose (0.764 g). Mixed thoroughly. Added crosslinked sodium carboxymethyl cellulose (0.512 g) and mixed thoroughly. Compressed to tablet (337 mg). Tablets were coated with Colorcon® Opadry® premixed enteric coating. Coated Tablets were tested by submerging in 1.0 N HCl for 30 minutes without any visible appearance changes.

Formulation

| | | | |
|---|---|---|---|
| Material | Ibuprofen/chitosan | lactose | crosslinked sodium carboxymethyl cellulose |
| % | 75% | 15% | 10% |
| Weight | 3.831 g | 0.764 g | 0.512 g |

### Example 3: Formulation of Reference #1 for Mucus Adhesive Ibuprofen/Chitosan (NW1901-80)

Added chitosan (2.03 g) to water (140 mL). To the slurry added 1.0 N HCl (10 mL). Stirred for 30 minutes and a clear solution was obtained. Diluted 1.0 N NaOH (10 mL) to 40 mL and dropwise added to the above chitosan solution. After stirring for 1 hour, separated chitosan polymer by centrifuging. Washed the polymer with water and hydrated chitosan (84.770 g) was obtained. Added water (40 mL) and ibuprofen (0.94 g) and stirred for 3 hours. All solid ibuprofen was dissolved. Added 1.0N HCl (11 mL), stirred for 3 hours. Evaporated water in 65° C. oven. Solid ibuprofen/chitosan material (3.32 g) was obtained and milled into powder with pestle and mortar.

Weighed the ibuprofen/chitosan material prepared above (3.070 g) and lactose (1.187 g). Mixed thoroughly. Added crosslinked sodium carboxymethyl cellulose (0.474 g) and mixed thoroughly. Compressed to tablet (302 mg). Tablets were coated with Colorcon® Opadry® enteric coating. Enteric coated tablets were tested by submerging in 1.0 N HCl for 30 minutes without any visible appearance changes.

| | | | |
|---|---|---|---|
| Material | ibuprofen/chitosan | lactose | crosslinked sodium carboxymethyl cellulose |
| % | 65% | 25% | 10% |
| Weight | 3.070 g | 1.187 g | 0.474 g |

### Example 4: Formulation of Reference #2 Mucus Adhesive Ibuprofen/Chitosan NW1901-80)

Added chitosan (2.01 g) to water (140 mL). To the slurry added 1.0 N HCl (11 mL). Stirred for 30 minutes and a clear solution was obtained. Evaporate water in 65° C. oven. Solid material (2.12 g) was obtained and milled into powder with pestle and mortar. Added ibuprofen (0.92 g) and mixed thoroughly. The powder mixture was used for preparation of reference #2 tablets.

Weighed the ibuprofen/chitosan mixture material prepared above (2.862 g) and lactose (1.118 g). Mixed thoroughly. Added crosslinked sodium carboxymethyl cellulose (0.441 g) and mixed thoroughly. Compressed to tablet (281 mg). Tablets were coated with Colorcon® Opadry® enteric coating. Enteric coated tablets were tested by submerging in 1.0 N HCl for 30 minutes without any visible appearance changes.

| | | | |
|---|---|---|---|
| Material | ibuprofen/chitosan | lactose | crosslinked sodium carboxymethyl cellulose |
| % | 65% | 25% | 10% |
| Weight | 2.862 g | 1.118 g | 0.441 g |

### Example 5: Preparation of Mucus Adhesive Levodopa Ethyl Ester/Hyaluronic Acid Material (NW2001-12)

Mixed levodopa ethyl ester (1.15 g) with hyaluronic acid (2.13 g). Added water (4.4 mL) at 0° C. with an ice bath and stirred the mixture thoroughly. Evaporated water at ambient temperature under vacuum. After milling with mortar and pestle, and sieving, powder material (2.77 g) was obtained. Added lactose powder (1.38 g), mixed well, then granulated by spraying 6.5% lactose solution. After drying and sieving, levodopa ethyl ester/hyaluronic acid material (3.99 g) was obtained.

### Example 6: Preparation of Mucus Adhesive Berserazide/Hyaluronic Acid Material (NW2001-11-39)

Mixed berserazide (0.96 g) with hyaluronic acid (1.56 g). Added water (4.0 mL) at 0° C. with an ice bath and stirred the mixture thoroughly. Evaporated water at ambient temperature under vacuum. After milling with mortar and pestle, and sieving, powder material (2.39 g) was obtained. Added lactose powder (1.19 g), mixed well, then granulated by spraying 6.5% lactose solution. After drying and sieving, berserazide/hyaluronic acid material (3.47 g) was obtained.

### Example 7: Formulation of Mucus Adhesive Levodopa Ethyl Ester/Benserazide/Hyaluronic Acid (NW2001-23-48; NW2001-26-7)

Weighed levodopa ethyl ester/hyaluronic acid material (3.99 g) obtained in Example 5 and benserazide/hyaluronic acid material (1.59 g) obtained in Example 6 into a container and mixed thoroughly. Weighed the above mixture (4.635 g) and microcrystalline cellulose (2.896 g), mixed thoroughly. Added crosslinked sodium carboxymethyl cellulose (0.365 g) and mixed thoroughly. Compressed to tablet (525 mg). Tablets were coated with Colorcon opadry enteric coating. Tablets were tested by submerging in 1.0 N HCl for 30 minutes without any visible appearance changes.

Formulation

| | | | |
|---|---|---|---|
| Material | levodopa ethyl ester/hyaluronic acid:benserazide/hyaluronic acid (4:1) | MCC | crosslinked sodium carboxymethyl cellulose |
| % | 59% | 36% | 5% |
| Weight | 4.635 g | 2.896 g | 0.365 g |

### Example 8: Preparation of Levodopa /Hyaluronate Sodium Material (NW2001-01-18)

Mixed levodopa (2.85 g) with hyaluronate sodium (4.01 g). Added water and stirred the mixture thoroughly until a thick viscus liquid was obtained. Evaporated water in a 50° C. oven. After milling with mortar and pestle, and sieving, powder material (5.86 g) was obtained. Added lactose powder (2.07 g), mixed well, then granulated by spraying water. After drying, added more lactose powder (1.98 g), mixed well, then granulated by spraying water. After drying in a 50° C. oven and sieving, levodopa/hyaluronate sodium material (9.79 g) was obtained.

### Example 9: Preparation of Benserazide HCl/Hyaluronate Sodium Material (NW2001-01-32)

Mixed benserazide HCl (1.06 g) with hyaluronate sodium (2.00 g). Added water and stirred the mixture thoroughly until a thick viscus liquid was obtained. Evaporated water in a 50° C. oven. After milling with mortar and pestle, and sieving, benserazide HCl/hyaluronate sodium powder material (2.97 g) was obtained. Added lactose powder (0.99 g), mixed well, then granulated by spraying water. After drying, added more lactose powder (1.03 g), mixed well, then granulated by spraying water. After drying in a 50° C. oven and sieving, benserazide HCl/hyaluronate sodium material (4.87 g) was obtained.

### Example 10: Formulation of Reference Levodopa/Benserazide HCl/Hyaluronate Sodium (NW2001-25)

Weighed levodopa/hyaluronate sodium material (9.79 g) obtained in Example 8 and benserazide HCl/hyaluronate sodium material (3.68 g) obtained in Example 9 into a container and mixed thoroughly. Weighed the above mixture (6.176 g) and microcrystalline cellulose (2.842 g), mixed thoroughly. Added crosslinked sodium carboxymethyl cellulose (0.495 g) and mixed thoroughly. Compressed to tablet (473 mg). Tablets were coated with Colorcon Opadry enteric coating. Tablets were tested by submerging in 1.0 N HCl for 30 minutes without any visible appearance changes.

Formulation

| | | | |
|---|---|---|---|
| Material | levodopa/hyaluronate sodium:benserazide HCl/hyaluronate sodium (4:1) | MCC | crosslinked sodium carboxymethyl cellulose |
| % | 65% | 30% | 5% |
| Weight | 6.176 g | 2.842 g | 0.495 g |

### Example 11 Animal Tests

### Dog Preparation

Regular Beagle dogs, body weight 12 kg, age 12 months old, were purchased and quarantined for 7 days. One dog was reserved for standby and the rest of dogs were used for the test. The test facility was maintained according to the following standard: temperature 20-26° C., humidity 40%-70%, fresh air exchange 8 times/hour, one dog per cage (L×W×H=90 cm×100 cm×90 cm). The dogs were fed with popped granulated adult dog food freely and the drinking water was supplied freely with a bite and press drinking water distributor. Before dosing, a blank blood sample (3 mL) was taken at the rear legs. Dosage for beagle dog was calculated according to “the technical guidelines for non-clinical pharmacokinetic study of drugs”, based on the body surface area conversion between an average human and a beagle dog, and considering the literature published and the special need of the investigation.

### Preparation of Plasma Samples

Ibuprofen: Sample time points were: 0.0, 0.5, 1, 2, 3, 4, 6, 8, 12, 24 hours. The plasma sample was subjected to 3000 G centrifuge for 10 minutes. Supernatant solution was taken and stored at -80° C. for future analytical work. Accurately transferred 2 µL of internal standard solution into a 10 mL centrifuge tube and blow N₂ to dryness. Added plasma sample (1 mL), shaken for a while. Added 10% trichloroacetic acid (0.5 ml) and mixed well. Added dichloromethane/isopropanol (50/2) (5 ml) and vertexed for 10 min. Centrifuged, sampled the organic phase (bottom layer) and washed with hydrochloric acid and ultra-pure water for 5 min, respectively. Centrifuged and isolated the organic phase, evaporated solvent in water bath. Dissolved the residue with 100 µ l mobile phase and injected 30 µl for HPLC analysis.

Levodopa ethyl ester/benserazide: Sample time points were: 0, 1, 2, 3, 4, 6, 8, 10, 12, 16, 20, 24 hours. Plasma sample (2 ml) was centrifuged at 4200 RPM at 8° C. for 10 min. The supernatant layer of the plasma was isolated and the same volume of 6% perchloric acid solution was added. After shaken for 2 min, the sample was centrifuged at 10000 RPM for 15 min. The supernatant solution was filtered with a 0.22 µm microporous membrane filter, and 20 µl was injected for HPLC analysis.

### HPLC Parameters

Ibuprofen: Column: lichrosorb RP-18 (150 mm × 4.6 mm, 5 µ m). Pre-column (50 mm × 4.6 mm, ID): octade-cyl-sil-x - II, dry filling. The mobile phase was methanol:0.1 M sodium acetate buffer (pH 5.0) 60:40. Detection wavelength: 230 nm. Column temperature ambient (21 ± 2) °C.

Levodopa ethyl ester/benserazide: Column: C18 column (250 mm × 4.6 mm, 5 µm), mobile phase: methanol (A) - 0.1% trifluoroacetic acid aqueous solution (B), gradient elution: 0-2 min 98% B, 2-25 min 98% - 82% B, 25-40 min 82% - 98% B, flow rate: 0.5 ml / min, column temperature: 30° C., injection volume: 20 µ L, detection wavelength: 220 nm

### Example 12: Dog Model Drug Release of Mucus Adhesive Ibuprofen/Chitosan Formulation

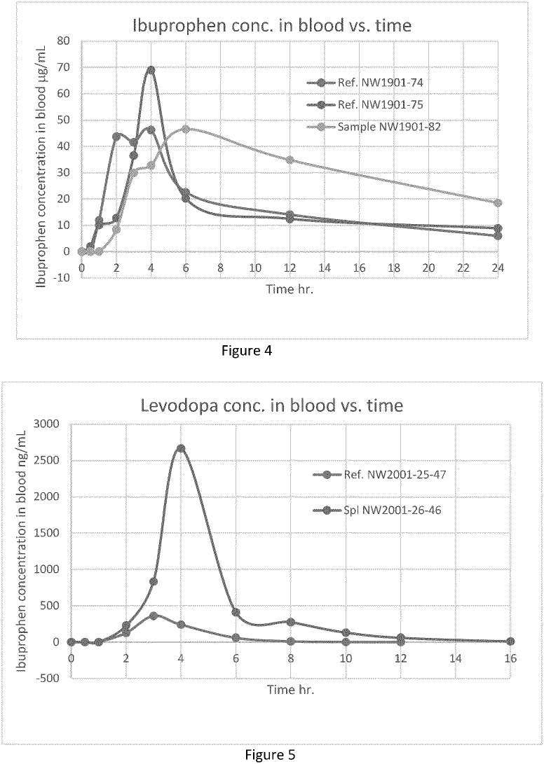

FIG. 4 is the ibuprofen blood concentration vs. time profile. As shown by the graph, the AUC of the mucus adhesive drug delivery is more than 70% greater than the AUC of the 2 reference conventional formulations (Reference 1 and Reference 2 as discussed above). Tmax of the mucus adhesion drug delivery is at 6 hours while the Tmax for the 2 reference conventional formulations is at 4 hours. There is a 2-hour delay. The mucus adhesive drug delivery is generally more extended than the 2 reference formulations. In terms of blood concentration, it can be seen that the mucus adhesive drug delivery provided the same ibuprofen concentration at 24-hour time point as the reference did at 6-hour time point. Thus, it can be claimed that the mucus adhesive drug delivery has extended the drug release from 6 hours to 24 hours. The increase was 3 folds.

### Example 13: Dog Model Drug Release of Mucus Adhesive Levodopa Ethyl Ester/Benserazide/Hyaluronic Acid Formulation

FIG. 5 shows levodopa blood concentration vs. time profile. As shown by the graph, there is a huge difference in AUC between the mucus adhesive drug delivery and the reference formulation. The AUC of the mucus adhesive drug delivery is 5.8 folds greater than that of the reference formulation. The Tmax of reference conventional drug delivery is at 3 hours while the Tmax of new mucus adhesion drug delivery is at 4 hours, one-hour delay. In terms of blood concentration, it can be seen that the mucus adhesive drug delivery provided the same levodopa concentration at 12-hour time point as the reference did at 6-hour time point. Thus, it can be claimed that the mucus adhesive drug delivery has extended the drug release by roughly 1-fold. This trend was hold true when the mucus adhesive drug delivery was at 8-hour time point as the reference did at 4-hour time point. The increase was one-fold.

The invention is not limited by the embodiments described above which are presented as examples only but can be modified in various ways within the scope of protection defined by the appended patent claims.

What is claimed is:

1. A composition comprising an effective amount of a biologically active agent bonded with a mucoadhesive polymer ionically or covalently or via metal coordination bonding, whereby after the composition is administered to a subject, the biologically active agent exerts its pharmaceutical effect while retaining on mucus of the animal.

2. The composition of claim 1 wherein the effective amount of biologically active agent bonded with a mucoadhesive polymer ionically or covalently or via metal coordination bonding is compressed into tablets with at least one excipient, and the tablets are coated with an enteric coating material so that the pharmaceutically active compound is gradually released in an intestine of the subject and absorbed through an intestine wall into blood stream of the subject in presence of NaCl or other ionic compound or via hydrolysis in the body fluid of the subject.

3. The composition of claim 1 wherein the biologically active agent comprises at least one functional group selected from the group consisting of amine, carboxyl, hydroxyl, sulfonic functional group —SO₃H, cationic metal, and combinations thereof, and the mucoadhesive polymer comprises at least one corresponding functional group selected from the consisting of carboxyl, hydroxyl, amine, sulfonic functional group —SO₃H, and combinations thereof so that the pharmaceutically active compound is bonded with the mucoadhesive polymer ionically or covalently or via metal coordination.

4. The composition of claim 1 wherein the biologically active agent is ionically bonded with the mucoadhesive polymer, and the pharmaceutically active compound is released in the body of the animal via metastasis.

5. The composition of claim 4 wherein the biologically active agent comprises at least one functional group selected from the group consisting of amine, carboxyl, sulfonic functional group —SO₃H, and combinations, and the mucoadhesive polymer comprises at least one corresponding functional group selected from the consisting of carboxyl, amine, sulfonic functional group —SO₃H, and combinations.

6. The composition of claim 1 wherein the biologically active agent is covalently bonded with the mucoadhesive polymer, and the pharmaceutically active compound is released in the body of the animal via hydrolysis.

7. The composition of claim 1 wherein the mucoadhesive polymer is selected from the group consisting of poly(methylvinylether co-methacrylic acid), poly(acrylic acid-co ethylhexylacrylate), copolymer of acrylic acid and poly ethylene glycolpolymer, copolymer of acrylic acid or methacrylic acid, carbopol, polycarbophil, carbomer, pectin, alginic acid, hyaluronic acid, chitosan, tragacanth gum, karaya gum, xanthan gum, carboxymethylcellulose, polyvinylamine, polyallylamine, polyethyleneimine, and combinations thereof.

8. The composition of claim 1 wherein the biologically active agent is selected from the group consisting of ibuprofen, lanthnum chloride, levodopa ethyl ester, aspirin, glutathione, valsartan, quetiapine, duloxetine, oxymorphone, and combinations thereof.

9. The composition of claim 1 comprising less than 50% by molar amount of the biologically active agent in free form relative to the biologically active agent bonded with the mucoadhesive polymer by molar amount.

10. The composition of claim 1 wherein the mucus adhesive materials bonded with drug molecules has a diameter of less than 3 mm.

11. The composition of claim 1 comprising no greater than 30% by molar amount of the biologically active agent in free form relative to the biologically active agent bonded with the mucoadhesive polymer by molar amount.

12. The composition of claim 1 wherein the biologically active agent has a half-life of no greater than 2 hours or a bioavailability of less than 80% due to missing out absorption window of GI tract, and the composition has an extended time of delivery of the biologically active agent for more than 12 hours.

13. The composition of claim 1 being formulated in a solid dosage form.

14. The composition of claim 13 wherein the solid dosage form is a fast disintegration form and further comprises a non-ionic filler excipient and a disintegrant.

15. A method of extending release time of a biologically active agent and increase bioavailability of the biologically active agent in a subject comprising:
preparing the composition of claim 1; and
administering the composition to a subject in an effective amount.

16. The method of claim 1 wherein the administering comprising delivering the composition through mucosa of mouth, nose, stomach, intestine, and/or or eye of the subject.

17. A method of making the composition of claim 1 comprising:
reacting an effective amount of the biologically active agent with mucoadhesive polymer in water or a non-aqueous solvent via acid-base reaction, metathesis reaction, or esterification reaction to make the pharmaceutically active compound bonded with the mucoadhesive polymer.
